(19) United States
(12) Patent Application Publication
(10) Pub. No.: US 2024/0266032 A1
(43) Pub. Date: Aug. 8, 2024

(54) MEDICAL DATA-PROCESSING APPARATUS, SYSTEM, AND METHOD

(71) Applicant: CANON MEDICAL SYSTEMS CORPORATION, Tochigi (JP)

(72) Inventors: Daigo OKUHATA, Sakura (JP); Takahiko NISHIOKA, Otawara (JP); Naoki MATSUMOTO, Sakura (JP)

(73) Assignee: CANON MEDICAL SYSTEMS CORPORATION, Tochigi (JP)

(21) Appl. No.: 18/430,743

(22) Filed: Feb. 2, 2024

(30) Foreign Application Priority Data
Feb. 3, 2023 (JP) 2023-015459

(51) Int. Cl. G16H 30/40; G16H 30/20

(52) U.S. Cl. CPC G16H 30/40; G16H 30/20

(57) ABSTRACT

A medical data-processing apparatus according to an embodiment includes a memory and processing circuitry. The memory stores definition information in which input data to be input and output data to be output are set per execution state of a workflow, for each of multiple analysis applications included in the workflow, or for each of multiple processes executed in each of the analysis applications. The processing circuitry executes the workflow based on the definition information, and performs at least either one of processing out of processing to switch sources of input data to be input to one analysis application out of the multiple analysis applications or input data to be input to one process out of the multiple processes, and processing of changing execution order of the multiple analysis applications or execution order of the multiple processes, according to an execution state of the workflow.

### CROSS-REFERENCE TO RELATED APPLICATIONS

This application is based upon and claims the benefit of priority from Japanese Patent Application No. 2023-015459, filed on Feb. 3, 2023; the entire contents of which are incorporated herein by reference.

### FIELD

Embodiments described herein relate generally to a medical data-processing apparatus, a system, and a method.

### BACKGROUND

A medical data-processing apparatus that automatically executes an analysis application, such as artificial intelligence (AI) with respect to digital imaging and communications in medicine (DICOM) data acquired from various kinds of medical diagnostic-imaging apparatuses has been available. DICOM data is data that conforms to the DICOM standard. The medical data-processing apparatus outputs an analysis result in such a manner that a user can view the analysis result. In the present specification, hereinafter, “analysis application” may be abbreviated as “app”.

The medical data-processing apparatus executes a workflow that includes multiple applications using DICOM data. For example, the medical data-processing apparatus executes the workflow based on definition information in which execution order of the apps, various kinds of conditions at the time of executing the respective apps, and the like are defined. There is a case in which, for example, an execution result (processing result) of one app is input to the other app, and the other app executes using the input processing result.

FIG. 1 is a diagram for explaining an example of processing performed by a conventional medical data-processing apparatus. For example, in definition information, it is defined that after execution of app A is completed, that is, when execution of app A did not fail, app B is executed. Moreover, in the definition information, it is defined that app A outputs result A′ of processing in app A to app B, and app B performs processing using result A′, and outputs result B of processing in app B. When executing a workflow based on the definition information as described, as illustrated in FIG. 1, the medical data-processing apparatus first executes app A, and after execution of app A is completed, executes app B using result A′ of processing in app A. Thus, result B of processing in app B is output.

For example, app A is an app that generates a perfusion image by using multiple pieces of chronological three-dimensional CT image data (four-dimensional CT image data) as the DICOM data. Moreover, app B is an app that performs a predetermined analysis (for example, analysis relating to cerebral infarction) for large vessel occlusion (LVO) of a subject by using the result A′. App A generates a perfusion image as result A of processing, and selects a three-dimensional CT image data with the highest concentration of contrast agent administered to a subject from among multiple pieces of three-dimensional CT image data, as the result A′ of processing to be output to app B. App B then performs a predetermined analysis for LVO using the three-dimensional CT image data with the highest concentration of contrast agent that has been output from app A, and outputs an analysis result as the result B.

Other examples of multiple apps included in a workflow will be explained. For example, the workflow includes an app that generates a diffusion weighted image (DWI) using magnetic resonance (MR) data, an app that generates a perfusion weighted image (PWI) using MR data, and an app that performs mismatch analysis to acquire a mismatch between the DWI and the PWI using the DWI and PWI generated by these two apps.

There is a case in which execution of a preceding app, which generates data (input data) to be input and used in a subsequent app, fails. In such a case, an error occurs, and the workflow process is terminated in the middle. As a result, subsequent processes are not executed, and a user cannot receive an expected result. Moreover, when multiple apps are executed in parallel, there is a case in which execution of either one out of the apps fails. In this case also, an error occurs and the workflow process is terminated in the middle. As a result, subsequent processes are not executed, and the user cannot receive an expected result. In the following, three examples when these errors occur will be explained.

The first example is a case in which although data to be used in a subsequent app is generated before an error occurs, the generated data cannot be used. FIG. 2 is a diagram for explaining the first example when an error occurs. As illustrated in FIG. 2, process A is executed in app A. In process A, after process A-1 is executed, process A-2 is executed. By process A-1, result A′ to be used in app B is generated, and by process A-2, result A is generated.

As illustrated in FIG. 2, there is a case in which execution of process A-2 fails even when the execution of process A-1 has been completed and result A′ to be used in app B has been generated. In this case, it is determined that the execution of app A has failed and an error has occurred. As described above, in the definition information, it is defined that app B is executed when execution of app A did not fail. That is, in the definition information, it is defined that app B is not executed if execution of app A fails. Therefore, a medical data-processing apparatus that executes the workflow stops execution of processing at a point when an error occurs, and does not perform subsequent processing. That is, app B is not to be executed. As described, there is a case in which the workflow process is terminated without executing app B even though result A′ to be used in app B has been generated.

The second example is a case in which although app A could not generate data to be used in app B subsequent to app A because an error has occurred in app A, but another app C compatible with app A has generated data to be used in app B. In this case, the data generated by app C is not used in app B. For example, when execution of app A fails, a medical data-processing apparatus stops execution of processing at the time when execution of app A fails and an error occurs, and does not execute subsequent processing. As described, even when data to be used in app B has been generated by app C, the workflow process can be stopped. Depending on a type or configuration of an app, it may be impossible to divide a single app into multiple apps by dividing a process executed in the single app into multiple processes.

The third example will be explained. For example, suppose that two apps, one app A and one app B, are executed in parallel and have executed common preprocessing, app A performs process A using a result of the preprocessing executed by app A, while app B performs process B using a result of the preprocessing executed by app B. In this case, even when the preprocessing in app A has failed, the result of the preprocessing to be used in process A of app A is generated by app B. However, a medical data-processing apparatus that executes the workflow stops execution of processing at the time when execution of the preprocessing of app A fails and an error occurs, and does not execute subsequent processing. As described, even when a result of preprocessing to be used in process A of app A has been generated by app B, execution of the workflow can be stopped.

Therefore, based on these facts, also when the workflow process can be continued to be executed even if an error has occurred, the workflow process can be stopped. Accordingly, it is desired to avoid termination of the workflow process even when an error occurs. That is, it is desired that the workflow be appropriately executed.

Moreover, even when a user desires to grasp only result of a subsequent app, a preceding app continues to operate also after outputting data to be used in processing of the subsequent app to the subsequent app and, therefore, resources of a processor, a memory, and the like of a central processing unit (CPU) of the medical data-processing apparatus can be consumed unnecessarily. FIG. 3 is a diagram for explaining an example when resources are consumed unnecessarily. As illustrated in FIG. 3, app A continues to execute processing as indicated by a range surrounded by a broken line frame in FIG. 3 even after generation of result A′ to be used in app B and output of result A′ to app B have been completed. This is because app A itself is an app that outputs result A of processing finally. App B cannot execute unless execution of app A is completed. In this case, the user desires to grasp result B of processing of app B, not result A of processing of app A. Therefore, for the user, it is considered unnecessary for App A to continue execution of processing after it outputs result A′. Therefore, it is desired that unnecessary consumption of resources be suppressed. That is, it is desired that the workflow be appropriately be executed.

### BRIEF DESCRIPTION OF THE DRAWINGS

FIG. 1 is a diagram for explaining an example of processing performed by a conventional medical data-processing apparatus;

FIG. 2 is a diagram for explaining a first example of a case in which an error occurs;

FIG. 3 is a diagram for explaining an example in which resources are unnecessarily consumed;

FIG. 4 is a diagram illustrating a configuration example of a medical data-processing system and a medical data-processing apparatus according to a first embodiment;

FIG. 5 is a diagram explaining an example when definition information according to the first embodiment is changed;

FIG. 6 is a diagram illustrating an example of a workflow after a change and input data input to process A′ that are displayed on at least one of a display and a terminal in the first embodiment;

FIG. 7 is a flowchart illustrating a flow of processing performed by respective processing functions included in processing circuitry of the medical data-processing apparatus according to the first embodiment;

FIG. 8 is a diagram for explaining an example when definition information according to a modification of the first embodiment is changed;

FIG. 9 is a diagram for explaining an example when definition information according to a second embodiment is changed;

FIG. 10 is a diagram for explaining an example of processing performed by an execution function according to the second embodiment;

FIG. 11 is a diagram illustrating an example of input data input to app D that is displayed on at least one of a display and a terminal in the second embodiment;

FIG. 12 is a diagram illustrating an example of definition information before a change according to a third embodiment;

FIG. 13 is a diagram illustrating an example of the definition information after a change according to the third embodiment; and

FIG. 14 is a diagram illustrating an example of an acceptance screen according to a fourth embodiment.

### DETAILED DESCRIPTION

One of problems to be solved by embodiments disclosed in the present specification and drawings is to execute a workflow appropriately. However, the problems to be solved by the embodiments disclosed in the present specification and drawings are not limited to the problems described above. Problems corresponding to respective effects obtained by respective configurations described in the embodiments described later can also be considered as other problems.

A medical data-processing apparatus according to an embodiment includes a memory and processing circuitry. The memory stores definition information in which input data to be input and output data to be output are set per execution state of a workflow, for each of multiple analysis applications included in the workflow, or for each of multiple processes executed in each of the analysis applications. The processing circuitry executes the workflow based on the definition information, and performs at least either one of processing out of processing to switch sources of input data to be input to one analysis application out of the multiple analysis applications or input data to be input to one process out of the multiple processes, and processing of changing execution order of the multiple analysis applications or execution order of the multiple processes, according to an execution state of the workflow.

Hereinafter, embodiments and modifications of a medical data-processing apparatus, a system, and a method will be explained in detail with reference to the drawings. The medical data-processing apparatus, the system, and the method according to the present application are not limited to the embodiments and modifications described below. Moreover, the embodiments can be combined with other embodiments, modifications, or conventional techniques within a range not causing a contradiction in processing. Similarly, the modifications may be combined with embodiments, other modifications, or conventional techniques within a range not causing a contradiction in processing.

### First Embodiment

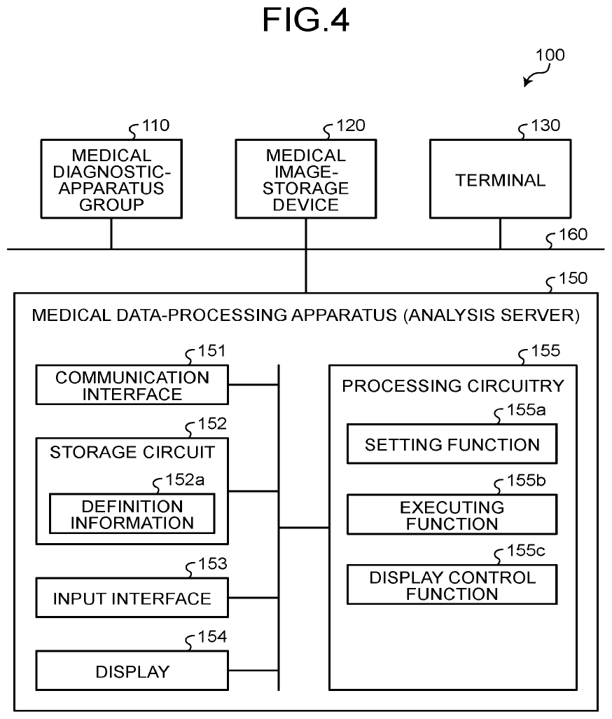

FIG. 4 is a diagram illustrating a configuration example of a medical data-processing system 100 and a medical data-processing apparatus 150 according to a first embodiment.

As illustrated in FIG. 4, the medical data-processing system 100 according to the present embodiment includes a medical diagnostic-apparatus group 110, a medical image-storage device 120, a terminal 130, and the medical data-processing apparatus 150. The respective devices are connected to one another through a network 160 in a communication enabled manner. The medical data-processing system 100 and the medical data-processing apparatus 150 according to the present embodiment are installed in, for example, medical facilities, such as hospitals and clinics, and supports diagnoses by a user, such as physicians. The medical data-processing system 100 is, for example, an example of a system.

The medical diagnostic-apparatus group 110 is a set of multiple medical diagnostic apparatuses. Examples of the medical diagnostic apparatus include an X-ray computed tomography (CT) apparatus, an ultrasound diagnostic apparatus, a magnetic resonance imaging (MRI) apparatus, an X-ray diagnostic apparatus, a positron emission tomography (PET) apparatus, and a single photon emission computed tomography (SPECT) apparatus, and the like.

The X-ray CT apparatus generates a CT image. The ultrasound diagnostic apparatus generates an ultrasound image. The MRI apparatus generates an MRI image. The X-ray diagnostic apparatus generates an X-ray image and an angiography image. The PET apparatus generates a PET image. The SPECT apparatus generates a SPECT image. The CT apparatus, the ultrasound image, the MRI image, the X-ray image, the angiography image, the PET image, and the SPECT image are all DICOM data, and are medical data and medical images.

The medical image-storage device 120 stores various kinds of medical images. Specifically, the medical image-storage device 120 acquires a medical image from the medical diagnostic-apparatus group 110 through the network 160, and stores the acquired medical image in a storage circuit within the medical image-storage device 120. Moreover, for example, the medical image-storage device 120 is implemented by a picture archiving and communication system (PACS) or the like, and stores the medical image in a DICOM-compliant format. The medical image-storage device 120 is implemented by a computer device, such as a server and a workstation.

The terminal 130 is a terminal used by a user, and displays various kinds of medical data. For example, the terminal 130 acquires various kinds of medical data, such as a medical image, an analysis result of analysis processing, and a processing result of image processing, from the medical image-storage device 120 and the medical data-processing apparatus 150 through the network 160. The terminal 130 displays the acquired medical data on a display equipped in the terminal 130. The display equipped in the terminal 130 is one example of a display unit. For example, the terminal 130 is implemented by a computer device, such as a workstation, a personal computer, and a tablet terminal.

The medical data-processing apparatus 150 performs various kinds of analysis processing and various kinds of image processing with respect to the medical data. For example, the medical data-processing apparatus 150 acquires medical data from the medical diagnostic-apparatus group 110 or the medical image-storage device 120 through the network 160. The medical data-processing apparatus 150 performs various kinds of analysis processing or various kinds of image processing with respect to the acquired medical data. The medical data-processing apparatus 150 is also referred to as analysis server or analysis device because it performs various kinds of analysis processing. For example, the medical data-processing apparatus 150 is implemented by a computer device, such as a server and a workstation. In the following explanation, a case in which a single unit of the medical data-processing apparatus 150 performs various kinds of processing explained below will be explained, but it may be configured such that multiple units of the medical data-processing apparatus 150 perform various kinds of processing explained below in a distributed manner.

As illustrated in FIG. 4, the medical data-processing apparatus 150 includes a communication interface 151, a storage circuit 152, an input interface 153, a display 154, and processing circuitry 155.

The communication interface 151 controls transmission and communication of various kinds of information and various kinds of data that are transmitted and received between the medical data-processing apparatus 150 and other devices connected to the medical data-processing apparatus 150 through the network 160. The communication interface 151 is connected to the processing circuitry 155. The communication interface 151 receives data transmitted by other devices. In this case, the communication interface 151 transmits the received data to the processing circuitry 155. Moreover, the communication interface 151 receives data transmitted by the processing circuitry 155. In this case, the communication interface 151 transmits the received data to other devices. For example, the communication interface 151 is implemented by a network car, a network adapter, a network interface controller (NIC), or the like.

The storage circuit 152 stores various kinds of data and various kinds of programs. The storage circuit 152 is connected to the processing circuitry 155. The storage circuit 152 stores data transmitted by the processing circuitry 155 under the control of the processing circuitry 155. Moreover, data stored in the storage circuit 152 is read by the processing circuitry 155. For example, the storage circuit 152 is implemented by a semiconductor memory device, such as a random access memory (RAM) and a flash memory device, a hard disk, an optical disk, and the like.

The storage circuit 152 stores definition information 152a. In the definition information 152a, definitions relating to execution of a workflow is indicated. For example, in the definition information 152a, execution order of multiple apps included in a workflow, various kinds of conditions at the time of executing the respective apps, and the like are defined. In the present embodiment, the medical data-processing apparatus 150 executes a workflow in accordance with the definition information 152a.

The input interface 153 accepts an input operation of various kinds of instructions and various kinds of information from a user, such as a physician. The input interface 153 is connected to the processing circuitry 155. The input interface 153 converts the operation accepted from the user into an electrical signal, to transmit to the processing circuitry 155. For example, the input interface 153 is implemented by a trackball, a switch button, a mouse, a keyboard, a touch pad that accepts an operation by being touched on an operating surface, a touch screen in which a display screen and a touch pad are integrated, a non-contact input interface using an optical sensor, a sound interface, or the like. In the present specification, the input interface 153 is not limited to that having a physical operating part, such as a mouse and a keyboard. For example, processing circuitry of an electrical signal that receives an electrical signal corresponding to an input operation from an external input device arranged separately from the apparatus, and transmits this electrical signal to the processing circuitry 155 is also included in examples of the input interface 153.

The display 154 displays various kinds of information and various kinds of data. The display 154 is connected to the processing circuitry 155. The display 154 displays various kinds of information and various kinds of data transmitted by the processing circuitry 155 under the control of the processing circuitry 155. For example, the display 154 is implemented by a liquid crystal display, a cathode ray tube (CRT) display, a touch panel, or the like. The display 154 is one example of the display unit.

The processing circuitry 155 controls the entire medical data-processing apparatus 150. For example, the processing circuitry 155 performs various kinds of analysis processing and various kinds of image processing according to the operation accepted from a user through the input interface 153. Moreover, for example, the processing circuitry 155 acquires medical data transmitted from the medical diagnostic-apparatus group 110 or the medical image-storage device 120. The processing circuitry 155 stores the acquired medical data in the storage circuit 152.

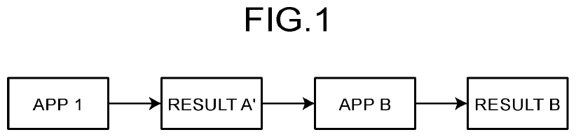

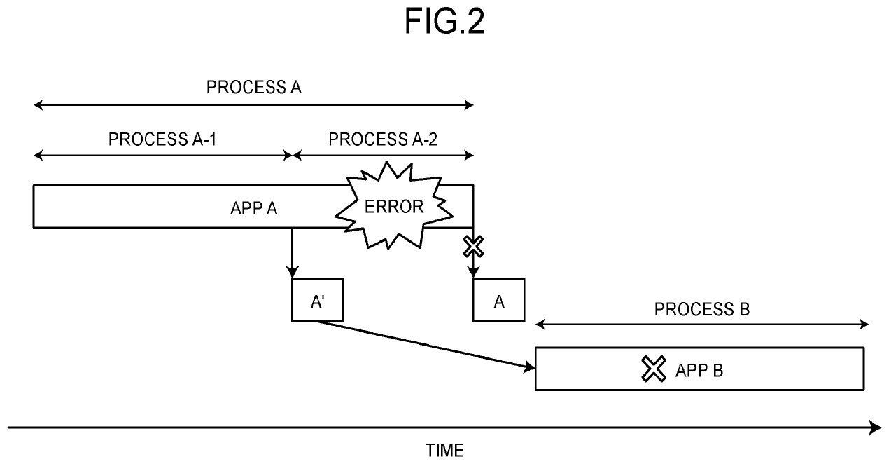

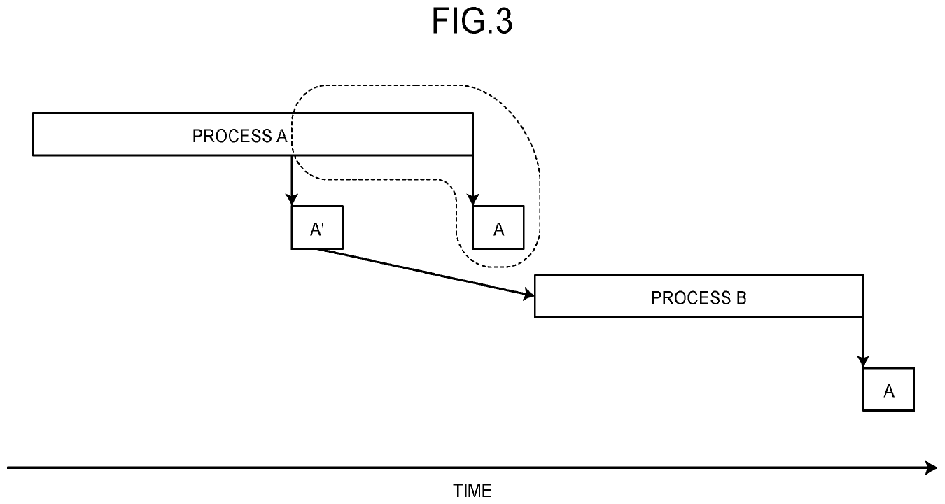

As illustrated in FIG. 1, the processing circuitry 155 includes a setting function 155a, an executing function 155b, and a display control function 155c. The setting function 155a is one example of a setting unit. The executing function 155b is one example of an executing unit. The display control function 155c is one example of a display control unit.

The processing circuitry 155 is implemented by, for example, a processor. In this case, the respective processing functions described above are stored in the storage circuit 152 in a form of computer-executable program (medical data-processing program). The processing circuitry 155 reads the respective programs stored in the storage circuit 152, and implements the respective processing functions corresponding to the respective programs by executing the read respective programs. In other words, the processing circuitry 155 have the respective processing functions illustrated in FIG. 4 when it is in a state in which it has read the respective programs.

The processing circuitry 155 may be constituted of multiple independent processors combined, and may be configured to implement the respective processing functions by executing the respective programs by the respective processors. Moreover, the respective processing functions of the processing circuitry 155 may be implemented by a single or multiple units of processing circuitry in a distributed or in an integrated manner. Furthermore, the respective processing functions of the processing circuitry 155 may be implemented by a combination of hardware, such as a circuit, and software. Moreover, an example in which the respective programs corresponding to the respective processing functions are stored in a single unit of the storage circuit 152 has been explained herein, but the respective programs may be stored in multiple storage circuits in a distributed manner. For example, it may be configured such that the respective programs corresponding to the respective processing functions are stored in multiple storage circuits in a distributed manner, and the processing circuitry 155 reads and executes the respective programs from the respective storage circuits.

As above, one example of configurations of the medical data-processing system 100 and the medical data-processing apparatus 150 has been explained. The medical data-processing system 100 and the medical data-processing apparatus 150 according to the present embodiment are configured to execute a workflow appropriately as explained below.

In the first embodiment, the executing function 155b executes a workflow including app A and app B. The app A and app B are executed in parallel. In the present embodiment, prior to execution of the workflow by the executing function 155b as described, definition relating to execution of the workflow indicated by the definition information 152a is changed by a user. Change of definition is a concept including addition of definition, deletion of at least a part of the definition, correction of at least a part of the definition, and the like.

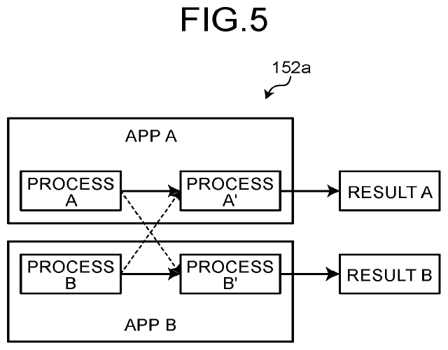

FIG. 5 is a diagram for explaining an example of a case in which the definition information 152a according to the first embodiment is changed. FIG. 5 illustrates an example of a definition registered in the definition information 152a. In the example in FIG. 5, a arrow indicates a flow of data. Moreover, a solid line arrow indicates a flow of data before subjected to a change by the user, that is, a flow of data that has originally been defined in the definition information 152a. Moreover, a broken line arrow indicates a flow of data added by the user, that is, a flow of data for which addition by the user has been accepted. In the following explanation, the definition information 152a before a flow of data is added by the user is referred to as “definition information 152a before a change”, and the definition information 152a to which a flow of data has been added by the user to the definition information 152a before a change is referred to as “definition information 152a after a change”.

FIG. 5 illustrates a definition indicated by the definition information 152a after a change. Therefore, the definition information 152a before a change will be explained. In the definition information 152a before a change, it is defined that app A and app B are executed in parallel. Moreover, in the definition information 152a before a change, it is defined that after process A is executed in app A, a result of processing of process A is input to process A′, process A′ is executed using the result of processing of process A, and result A of processing of process A′ is output by app A as result A of the process of app A. Furthermore, in the definition information 152a before a change, it is defined that in app B, after executing process B, a result of processing of process B is input to process B′, process B′ is executed using the result of processing of process B, and result B of processing of process B′ is output as result B of the process of app B by app B. In the definition information 152a before a change, these are originally defined.

Next, a case in which definition information 152a is changed by the user will be explained. The setting function 155a displays a definition indicated by the definition information 152a before a change on at least one of the display 154 and the terminal 130 in a representation of a workflow diagram. For example, the definition indicated by the definition information 152a before a change displayed on at least one of the display 154 and the terminal 130 is a definition obtained by deleting two broken line arrows from the definition indicated by the definition information 152a after a change illustrated in FIG. 5. The setting function 155a displays the definition information 152a such that various kinds of changes can be made by the user. The user can easily grasp various kinds of conditions, such as execution order of multiple apps and execution order of multiple kinds of processing performed in the respective apps, intuitively as the definition information 152a is displayed in a representation of a workflow diagram. In the example in FIG. 5, a case in which granularity of the diagram is an app unit, and a more detailed processing (algorithm) unit than the app unit is illustrated, but the granularity of the diagram is not limited thereto. As long as it is a unit generating one result, any unit may be used. Moreover, as long as a similar definition can be obtained, the displayed representation is not limited to a diagram.

A case in which process A and process B are compatible and, therefore, a result of processing of process A and a result of processing of process B are also compatible will be explained. In this case, for example, even when execution of process A has failed and a result of processing of process A is not input to process A′, as long as execution of process B has completed without fail, process A′ can be executed using a result of processing of process B.

Accordingly, the user sets a broken line arrow illustrated in FIG. 5 from process B to process A′, meaning that a result of processing of process B is input to process A′ if execution of process A fails by operating the input interface 153. Specifically, the setting function 155a sets a broken line arrow from process B to process A′ indicating that a result of processing of process B is input to process A′ if execution of process A fails, based on the operation of the user accepted through the input interface 153. Thus, in the definition information 153a, it is defined that if execution of process A fails, a result of processing of process B is input to process A′, and process A′ is executed using the result of processing of process B. More specifically, in the definition information 152a, an event of occurrence of an error in process A, and execution of process A′ by inputting a result of processing of process B to process A′ and using the result of processing of process B are registered in an associated manner.

Furthermore, the user sets a broken line arrow from process A to process B′ illustrated in FIG. 5 indicating that a result of processing of process A is input to process B′ if execution of process B fails, by operating the input interface 153. Specifically, the setting function 155a sets a broken line arrow from process A to process B′ indicating that a result of processing of process A is input to process B′ if execution of process B fails, based on the operation of the user accepted through the input interface 153. Thus, in the definition information 153a, it is defined that if execution of process B fails, a result of processing of process A is input to process B′, and process B′ is executed using the result of processing of process A. More specifically, in the definition information 152a, an event of occurrence of an error in process B, and execution of process B′ by inputting a result of processing of process A to process B′ and using the result of processing of process A are registered in an associated manner.

After the definition information 152a is thus changed, the executing function 155b executes the workflow in accordance with the definition information 152a after a change. For example, when an event of occurrence of an error in process A is detected, the executing function 155b executes processing associated with this event. Specifically, the executing function 155b inputs a result of processing of process B to process A′, and executes process A′ using the result of processing of process B. Moreover, when an event of occurrence of an error in process B is detected, the executing function 155b executes processing associated with this event. Specifically, the executing function 155b inputs a result of processing of process A to process B′, and executes process B′ using the result of processing of process A.

The event indicates an execution state of the workflow. That is, the executing function 155b executes the workflow based on the definition information 152a, and executes processing to switch sources of input data to be input to one process (process A′ or process B′) out of multiple processes depending on the execution state of the workflow. The executing function 155b switches sources of input data to be input to the one process, and inputs intermediate data of the other app (app B or app A) that is a different app from an app (app A or app B) executing one process, as input data to the one process. Moreover, the executing function 155b switches sources of input data to be input to one process, and inputs data of the other process (process B or process A) that is a different process from the one process, as input data to the one process. Furthermore, when an error occurs in execution of the workflow as the execution state of the workflow, the executing function 155b changes the workflow by switching the source of input data to be input to one process to a source in the event of occurrence of an error based on the definition information 152a.

Therefore, the setting function 155a described above sets input data to be input to the respective processes and output data to be output from the respective processes depending on the execution state of the workflow with respect to the definition information 152a. Moreover, the definition information 152a is information in which input data to be input and output data to be output are set for each execution state of a workflow for each of multiple processes to be executed by each of the multiple apps included in the workflow.

In the example illustrated in FIG. 5, the setting function 155a may display a data type of input data that is input to the respective processes (process A, process A′, process B, process B′), and that can be used for the process, and a data type of output data that is output from the respective processes on at least one of the display 154 and the terminal 130. The data type is, for example, two-dimensional image, three-dimensional image, CT image, n pieces of (n is a positive integer) images of specific phases of the heartbeat, and the like. Thus, the user can grasp the data type of necessary input data and output data for each process easily and, therefore, can perform settings of input data to be switched according to an execution state of the workflow easily.

Moreover, the setting function 155a may display an icon indicating requirements of input data (for example, whether it is a two-dimensional image or a three-dimensional image, resolution of the image, type of the image, whether it is a moving image or a still image, and the like) instead of the data type at a position corresponding to an input of the process to which the input data is input. Similarly, the setting function 155a may display an icon indicating requirements of output data (for example, whether it is a two-dimensional image or a three-dimensional image, resolution of the image, type of the image, whether it is a moving image or a still image, and the like) instead of the data type at a position corresponding to an output of the process to which the output data is output. The user can easily determine a output of which process should be connected to an input of which process, by viewing the icon.

Specific examples of process A, process A′, and result A of app A, and process B, process B′, and result B of app B according to the first embodiment will herein be explained. For example, data input to app A is a first DICOM series and second DICOM data, and data input to app B is also the first DICOM series and the second DICOM data. The first DICOM series is, for example, multiple chronological medical images in which a predetermined part of a subject is drawn. Moreover, a second DICOM series is multiple chronological medical images in which the predetermined part of the subject same as the predetermined part of the subject drawn in the first DICOM series are drawn. The first DICOM series and the second DICOM series are, for example, multiple medical images generated by the same medical diagnostic apparatus, but the imaging timing is different between the first DICOM series and the second DICOM series. The first DICOM series and the second DICOM series may be generated by different medical diagnostic apparatuses.

Both of processes, process A of app A and process B of app B, are a process for performing registration between the first DICOM series and the second DICOM series. Process A outputs a result of registration to process A′, which is a subsequent process to process A, as a result of processing of process A. Moreover, process B outputs a result of registration to process B′, which is a subsequent process to process B, as a result of processing of process B. As described, process A and process B are compatible, and a result of processing of process A and a result of processing of process B are also compatible.

Process A′ calculates a difference between the first DICOM series and the second DICOM series subjected to registration using the input result of registration, and outputs the calculated difference as result A. Moreover, process B′ superimposes the first DICOM series and the second DICOM series that have been subjected to registration by using the input result of registration, and outputs a result obtained by superimposition as result B.

As described, the medical data-processing system 100 and the medical data-processing apparatus 150 according to the first embodiment continue to perform subsequent processing in a form desired by the user even when execution of process A or process B fails. Therefore, according to the medical data-processing system 100 and the medical data-processing apparatus 150 according to the first embodiment, even when an error occurs, termination of processing of a workflow can be avoided in a form desired by the user. Accordingly, processes that have already been executed are not to be wasted, and the efficiency in execution of apps can be improved. Therefore, a workflow can be executed appropriately.

Moreover, manual re-execution of processing becomes unnecessary, and a load on the user can be reduced. Moreover, even if app A and app B are third-party apps, by visualizing intermediate processing of app A and app B and a flow of data, and by having the user set switching of data to be input to process A′ and process B′ in the event of occurrence of an event, a more detailed workflow can be constructed.

The setting function 155a may register not one processing but multiple processing associated with one event as occurrence of an error, and may assign priorities to the respective processing to be registered. The executing function 155b may be configured to determine, when the event of occurrence of an error is detected, whether execution of processing is possible sequentially from processing with the highest priority, and to execute processing determined to be possible to be executed.

The display control function 155c displays a workflow after a change (restructured workflow) and input data (intermediate data of app B or app A) input to process A′ or Process B′ when an event of termination of execution of the workflow is detected on at least one of the display 154 and the terminal 130. The event of termination of execution of the workflow is input by the user through the input interface 153 to the processing circuitry 155. When this event is input, the display control function 155c displays the workflow after a change and input data input to process A′ and process B′ on at least one of the display 154 and the terminal 130. When displaying the workflow after a change and the input data on the terminal 130, the display control function 155c transmits the workflow after a change and the input data to the terminal 130 through the communication interface 151. Having received the workflow after a change and the input data, the terminal 130 displays the received workflow after a change and input data on the display. The display control function 155c performs similar processing when displaying other data on the terminal 130 also.

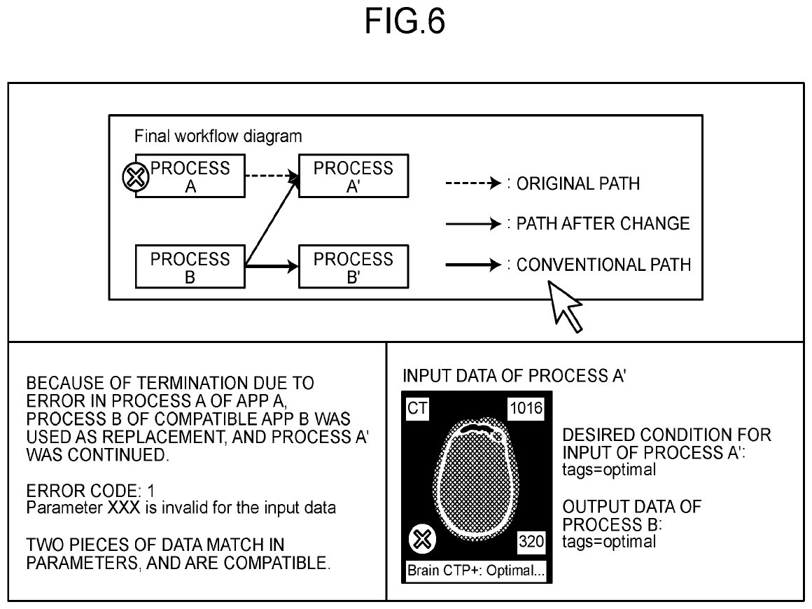

FIG. 6 is a diagram illustrating an example of the workflow after a change and the input data input to process A′ displayed on at least one of the display 154 and the terminal 130 in the first embodiment. As illustrated in FIG. 6, the display control function 155c displays a diagram of the workflow after a change. In the example in FIG. 6, a broken line arrow indicates an original flow of data (ORIGINAL PATH), and an arrow of a thin solid line indicates a flow of data after a change (PATH AFTER CHANGE), and an arrow of a thick solid line indicates a conventional flow of data (CONVENTIONAL PATH) in the diagram of the workflow after a change. Therefore, the user can easily grasp a flow of data in the workflow before a change and a flow of data in the workflow after a change intuitively from the displayed diagram of the workflow after a change. Moreover, the user can easily confirm if the workflow after a change is the intended workflow.

Furthermore, as illustrated in FIG. 6, the display control function 155c displays input data input to process A′ on at least one of the display 154 and the terminal 130. In the example in FIG. 6, the display control function 155c displays a medical image indicating a result of registration as input data. Therefore, the user can easily grasp data input to process A′ intuitively. Moreover, the user can confirm whether intended data is input to process A′ easily.

Furthermore, by displaying the diagram of the workflow after a change and the input data input to process A′, the user can easily confirm whether processing is performed properly. This leads to support the user to find a more effective workflow.

Moreover, as illustrated in FIG. 6, the display control function 155c displays an attribute of input data to be used in process A′ and an attribute of the input data actually output from process B and input to process A′ in a comparable manner on at least one of the display 154 and the terminal 130. In the example in FIG. 6, the display control function 155c displays “optimal” as an attribute of input data to be used in process A′. For example, tags are added to a medical image, and for the tags, various kinds of attributes are registered. Furthermore, the display control function 155c displays “optimal” as an attribute of input data that is actually output from process B and input to process A′. As described, the display control function 155c displays an attribute of input data to be used in one process (process A′) and an attribute of data of another process (process B) on at least one of the display 154 and the terminal 130. By checking the two attributes, the user can easily confirm whether intended data is input to process A′. In the case illustrated in FIG. 6, because the attributes of the two match, the user can confirm that intended data is input to process A′ easily.

When the attributes of two do not match, the user can determine that unintended data is input to process A′ easily. In this case, the display control function 155c may display an acceptance screen to accept an instruction from the user to change the workflow or to perform manual re-execution of process in which an error has occurred on at least one of the display 154 and the terminal 130. The medical data-processing apparatus 150 may perform processing in accordance with the instruction accepted from the user.

Furthermore, as illustrated in FIG. 6, the display control function 155c displays a message indicating what kind of change in the workflow has occurred from what kind of cause on at least one of the display 154 and the terminal 130. That is, the display control function 155c displays a message indicating the cause of the change of the workflow and details of the change of the workflow on at least one of the display 154 and the terminal 130. For example, the display control function 155c displays a message, “Because of termination due to an error in process A of app A, process B of compatible app B was used as a replacement, and process A′ was continued” as the message. Thus, the user can easily understand what kind of change has made to the workflow with what cause.

Moreover, as illustrated in FIG. 6, the display control function 155c displays an error code that easily enables the user to understand the cause of failure of execution of process A of app A on at least one of the display 154 and the terminal 130. In the example in FIG. 6, the display control function 155c displays “ERROR CODE: 1” and “Parameter XXX is invalid for the input data”. By this display, the user can easily understand the cause of failure of execution of process A of app A.

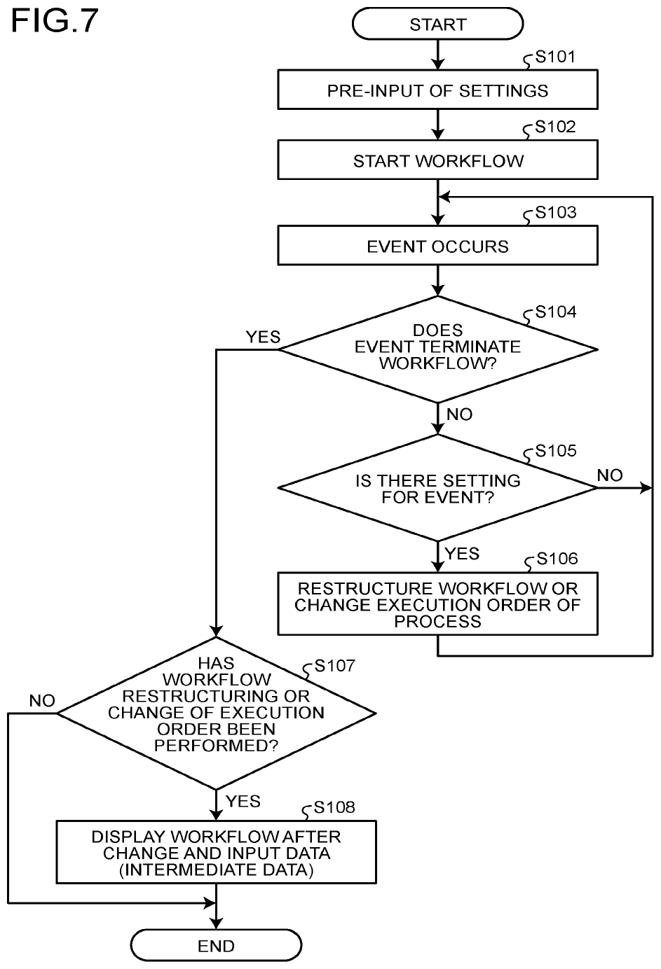

Next, a flow of processing performed by the medical data-processing apparatus 150 will be explained using FIG. 7. FIG. 7 is a flowchart illustrating a flow of processing performed by the respective processing functions of the processing circuitry 155 in the medical data-processing apparatus 150 according to the first embodiment.

Processing at step S101 and processing at step S102 and later illustrated in FIG. 7 are performed asynchronously. However, the processing at step S101 and the processing at step S102 and later may be performed in synchronization.

As illustrated in FIG. 7, first, the setting function 155a changes the definition information 152a based on an instruction of a user input through the input interface 153 (step S101).

The executing function 155b starts execution of a workflow (step S102). When an event occurs (step S103), the executing function 155b determines whether the occurred event is an event that terminates the execution of the workflow (step S104).

When the occurred event is not the event that terminates the execution of the workflow (step S104: NO), the executing function 155b determines whether predetermined processing is set for the occurred event (step S105). For example, at step S105, by determining whether predetermined processing is associated with the occurred event, whether predetermined processing is set for the occurred event is determined. For example, when predetermined processing is associated with the occurred event, the executing function 155b determines that predetermined processing is set for the occurred event. On the other hand, when predetermined processing is not associated with the occurred event, the executing function 155b determines that predetermined processing is not set for the occurred event.

In the case in which predetermined processing is not set for the occurred event (step S105: NO), when an event occurs again (step S103), the processing at step S104 and later is performed again. That is, each time an event occurs, the processing at step S104 and later is performed.

When predetermined processing is set for the occurred event (step S105: YES), the executing function 155brestructures the workflow or change execution order of processes by performing the predetermined processing set for the event (step S106), and when an event occurs again (step S103), the processing at step S104 and later is performed again.

On the other hand, when the occurred event is an event that terminates the execution of the workflow (step S104: YES), the executing function 155b terminates the execution of the workflow, and the display control function 155c determines whether the workflow has been restructured or the process execution order has been changed at step S106 (step S107).

When the workflow has been restructured or the process execution order has been changed (step S107: YES), the display control function 155c displays a workflow after a change and input data (intermediate data) on at least one of the display 154 and the terminal 130 (step S108), and the processing illustrated in FIG. 7 is ended. Moreover, when the workflow is not restructured and the process execution order is not changed (step S107: NO) also, the processing illustrated in FIG. 7 is ended.

As above, the medical data-processing system 100 and the medical data-processing apparatus 150 according to the first embodiment have been explained. According to the medical data-processing system 100 and the medical data-processing apparatus 150 according to the first embodiment, the workflow can be executed appropriately as described above.

In the first embodiment, a case in which the definition information 152a is information in which input data to be input and output data to be output depending on an execution state of a workflow are set for each of multiple processes executed in respective apps included in the workflow has been explained. However, the definition information 152a is not limited thereto. For example, the definition information 152a may be information in which input data to be input and output data to be output depending on an execution state of a workflow are set for each of apps included in the workflow. In this case, the executing function 155b executes the workflow based on the definition information 152a, and performs processing to switch sources of input data to be input to one app out of the apps depending on an execution state of a workflow. It will be explained with a specific example. The executing function 155b switches sources of input data to be input to one app, and inputs intermediate data of another app to the one app as the input data. To explain more specifically, the executing function 155b changes the workflow by switching the source of input data to be input to one app to a source in the event of occurrence of an error based on the definition information 152a, when an error in execution of the workflow occurs as the execution state of the workflow. The display control function 155c displays the input data input to the one app after the source is changed on at least one of the display 154 and the terminal 130.

### Modification of First Embodiment

In the first embodiment, a case in which the medical data-processing apparatus 150 switches sources of input data to be input to one app out of multiple apps or input data to be input to one process out of multiple processes depending on an execution state of a workflow has been explained. However, the medical data-processing apparatus 150 may select data to be used for a process from among multiple pieces of data stored in the storage circuit 152 depending on an execution state of a workflow, and may input the selected data to the process as input data. Such a modification will be explained as a modification of the first embodiment. In the explanation of the modification of the first embodiment, a point different from the first embodiment described above will be mainly explained, and explanation of configurations similar to those of the first embodiment described above may be omitted.

In the modification, the executing function 155b executes a workflow, for example, including a single app X. In the present modification, prior to execution of the workflow by the executing function 155b as described, a definition relating to execution of the workflow indicated in the definition information 152a will be changed by the user.

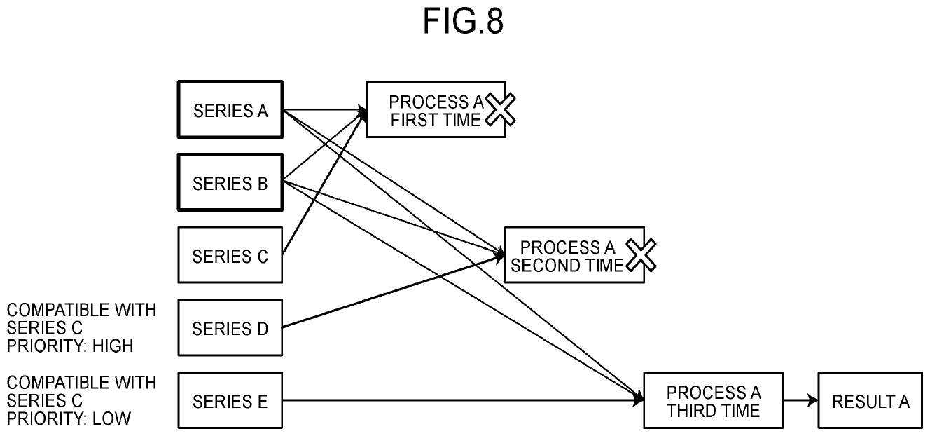

FIG. 8 is a diagram for explaining an example in which the definition information 152a according to the modification of the first embodiment is changed. In FIG. 8, one example of definition registered in the definition information 152a is illustrated. In the example in FIG. 8, an arrow indicates a flow of data. In FIG. 8, a definition indicated by the definition information 152a after a change is illustrated. Specifically, FIG. 8 illustrates the definition information 152a after a change in which an arrow indicating input of series D to process A, an arrow indicating input of series E to process A, and respective priorities of series D and series E are set. To explain series A to E, each of series A to E is multiple chronological medical images in which a predetermined part of a subject is drawn. However, series A to E have different imaging timing. Series A to E may be series generated by the same medical diagnostic apparatus, or may be series generated by not the same medical diagnostic apparatus. Series A to E are stored in the storage circuit 152 in advance.

To explain the definition information 152a before a change, in the definition information 152a before a change, it is defined that three series, series A, series B, and series C, are input to app X, and process A is executed using the three series, series A, series B, and series C. Moreover, in the definition information 152a before a change, it is defined that result A of processing of process A is output by app X. In the definition information 152a before a change, these are originally defined.

Next, a case in which the definition information 152a is changed by a user will be explained. The setting function 155a displays definitions indicated in the definition information 152a before a change in a representation of a diagram of a workflow on at least one of the display 154 and the terminal 130. The setting function 155a displays the definition information 152a in such a manner that various kinds of changes can be made by the user. The user can easily grasp various kinds of conditions, such as types of three series used for a process executed in app X intuitively, as the definition information 152a is displayed in a representation as a diagram of the workflow.

Series C and series D are compatible, and series C and series E are compatible also. Therefore, the executing function 155b can execute process A using series D instead of series C. Moreover, the executing function 155b can execute process A using series D instead of series C.

Therefore, as illustrated in FIG. 8, the user newly sets the definition information 152a such that process A is executed using series D or series E instead of series C when execution of process A using series A, series B, and series C fails. Specifically, the setting function 155a sets newly that when execution of process A using series A, series B, and series C fails, process A is executed using series D or series E instead of series C in the definition information 152a, based on the operation of the user accepted through the input interface 153. More specifically, the setting function 155a marks series D and series E as a series usable in place of series C.

In this case, the user sets the priority to each of the series D and series E by operating the input interface 153. This priority indicates which of series D and series E is used first when execution of process A using series C fails. The series having higher priority is used first. In the present modification, until the execution of process A is completed, series having higher priority are sequentially used. Specifically, the executing function 155b inputs series sequentially from the higher priority out of multiple series marked as usable in place of series C to process A, and executes process A using input series until the execution of process A is completed.

In the example in FIG. 8, a case I which the user sets “HIGH” as the priority of series D, and sets “LOW” as the priority of series E is illustrated. Specifically, the setting function 155a sets “HIGH” as the priority of series D, and sets “LOW” as the priority of series E based on the operation of the user accepted through the input interface 153. Thus, the priority of the series D becomes higher than the priority of series E. Accordingly, the series D is prioritized to be used than the series E.

By the processing described above, in the definition information 152a, it is defined that when execution of process A using series A, series B, and series C fails, series A, series B, and series D are newly input to process A and process A is executed using series A, series B, and series D, and when execution of process A using series A, series B, and series D fails, series A, series B, and series E are newly input to process A and process A is executed using series A, series B, and series E.

More specifically, in the definition information 152a, an event of occurrence of an error in process A executed using series A, series B, and series C, and execution of process A by inputting series A, series B, and series D to process A and by using series A, series B, and series D are registered in an associated manner.

Furthermore, in the definition information 152a, an event of occurrence of an error in process A executed using series A, series B, and series D, and execution of process A by inputting series A, series B, and series E and by using series A, series B, and series E are registered in an associated manner.

As described, in the definition information 152a, input data to be input to process A executed in an app included in the workflow for respective execution states of the workflow is set.

After the definition information 152a is thus changed, the executing function 155b executes the workflow in accordance with the definition information 152a after a change as illustrated in FIG. 8. For example, when an event of occurrence of an error in process A executed using series A, series B, and series C is detected, the executing function 155b executes processing associated with this event. Specifically, the executing function 155b inputs series A, series B, and series D to process A, and executes process A using series A, series B, and series D.

Furthermore, when an event of occurrence of an error in process A executed using series A, series B, and series D is detected, the executing function 155b performs processing associated with this event. Specifically, the executing function 155b inputs series A, series B, and series E to process A, and executes process A using series A, series B, and series E, and app X outputs result A of process A. As described, according to the present modification, even when an error occurs, process A can be continued to be executed.

As described above, the executing function 155b executes the workflow based on the definition information 152a, and switches input data to be input to process A depending to an execution state of the workflow.

Moreover, as described above, in the definition information 152a, first input data is set as input data to be input to process A when the execution state of the workflow is in a first execution state, and second input data replaceable with the first input data is set as input data to be input to process A when the execution state of the workflow is in a second execution state are set. The executing function 155b executes process A by inputting the first input data to process A when the execution state of the workflow is in the first execution state, and executes process A by inputting the second input data to process A when the execution state of the workflow is in the second execution state. The first execution state is, for example, a state in which an error occurs as a result of failure in execution of process A using series A, series B, and series C. Moreover, the second execution state is, for example, a state in which an error occurs as a result of failure in execution of process A using series A, series B, and series D. Furthermore, the first input data is, for example, three series including series A, series B, and series C. Moreover, the second input data is, for example, three series including series A, series B, and series D.

Furthermore, in the definition information 152a, multiple pieces of input data (for example, series D and series E) for respective execution states of the workflow are set with respect to process A, and priorities corresponding to the respective pieces of input data are set. The executing function 155b switches input data to be input to process A from among the multiple pieces of input data according to the priorities.

Specific examples of process A and result A according to the modification of the first embodiment will be explained. For example, process A is processing in which series C is used when performing registration between series A and series B. Specifically, in process A, based on series C, series C and series A are registered, and series C and series B are registered. If medical images included in series C are unclear, or when organ segmentation fails or the like, registration also fails. When the execution of process A is completed, process A outputs a result of registration as result A.

The technique according to the present modification is applicable to a workflow including multiple apps also. For example, a case in which app C that generates a perfusion image using a four-dimensional CT image, and app D that performs predetermined analysis (for example, analysis relating to cerebral infarction) of LVO using a result of app C are included in a workflow will be explained. Although app C generates a perfusion image as a final execution result, it selects three-dimensional CT image data with the highest concentration of contrast agent administered to a subject from among multiple pieces of three-dimensional CT image data as a result of processing output to app D, and outputs the selected three-dimensional CT image data to app D. App D then performs predetermined analysis for LVO using the three-dimensional CT image data with the highest concentration of contrast agent output from app C, and outputs an analysis result.

The executing function 155b can execute app D using not only three-dimensional CT image data output from app C, but also three-dimensional CT image data transmitted to the medical data-processing apparatus 150 from the X-ray CT apparatus. Therefore, when output of three-dimensional CT image data from app C to app D fails, the executing function 155b may execute app D using three-dimensional CT image data transmitted from the X-ray CT apparatus to the medical data-processing apparatus 150.

As described above, the medical data-processing system 100 and the medical data-processing apparatus 150 according to the modification of the first embodiment continues to execute process A in a form desired by the user even when execution of process A fails. Therefore, according to the medical data-processing system 100 and the medical data-processing apparatus 150 according to the modification of the first embodiment, even when an error occurs, termination of processing of a workflow can be avoided in a form desired by the user. Therefore, processes that have been executed are not wasted, and the efficiency in execution of an app can be improved. Accordingly, a workflow can be executed appropriately.

In the first embodiment and the modification of the first embodiment described above, a case in which the executing function 155b automatically restructures a workflow when an error occurs has been explained. However, the executing function 155b may automatically restructure a workflow before the workflow is executed. When the executing function 155b automatically restructures the workflow before the workflow is executed, the workflow is automatically restructured such that multiple pieces of output data output from multiple processes are input to a specific process. Thus, even when either one of the processes fails, the executing function 155b can execute the specific process. Therefore, it is possible to add redundancy to the workflow.

Moreover, when output data of process V aimed only to generate input data to be input to specific process U is input to specific process U as input data to be input to specific process U, it is possible to execute specific process U using output data of another process W in some cases. In this case, the executing function 155b may input the output data of process W to specific process U without executing process V, and may execute specific process U using the output data of process W. Thus, the overall processing load of the workflow can be reduced.

### Second Embodiment

The medical data-processing apparatus 150 may restructure a workflow according to a result that the user wishes to grasp in priority, to execute the restructured workflow. Accordingly, such an embodiment will be explained as a second embodiment. In the explanation of the second embodiment, a point different from the first embodiment described above will be mainly explained, and explanation of a configuration similar to that of the first embodiment may be omitted.

In the second embodiment, the executing function 155b executes a workflow including app C and app D. Prior to such execution of the workflow by the executing function, definition relating to execution of the workflow indicated in the definition information 152a is changed by the user.

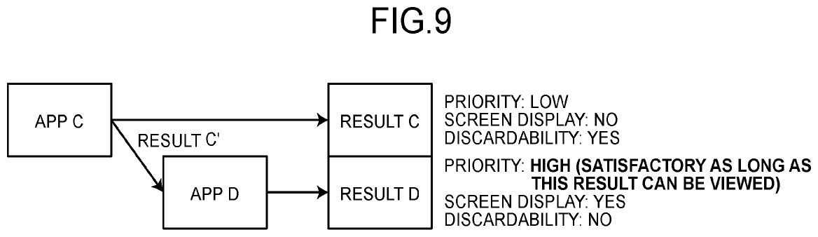

FIG. 9 is a diagram for explaining an example in which the definition information 152a according to the second embodiment is changed. In the example in FIG. 9, an arrow indicates a flow of data.

In FIG. 9, an example of a definition registered in the definition information 152a after a change is illustrated. Therefore, the definition information 152a before a change will be explained. In the definition information 152a before a change, it is defined that app C is executed. Moreover, in the definition information 152a before a change, it is defined that app D is executed after execution of app C is completed. Furthermore, in the definition information 152abefore a change, it is defined that app C outputs result (intermediate data) C′ of app C to app D, and also outputs final execution result C. Moreover, in the definition information 152a before a change, it is defined that app D is executed using result C′, and result D of the process of app D is output. In the definition information 152a before a change, these are originally defined.

Next, a case in which the definition information 152a is changed by the user will be explained. The setting function 155a displays the definition indicated in the definition information 152a before a change in a representation of a diagram of a workflow on at least one of the display 154 and the terminal 130. The setting function 155a displays the definition information 152a in such a manner that various kinds of changes can be made by the user. In the example in FIG. 9, a case in which granularity of the diagram is an app unit, but the granularity of the diagram is not limited thereto. As long as it is a unit generating one result, any unit may be used. Moreover, as long as a similar definition can be obtained, the displayed representation is not limited to a diagram.

A case in which the user desires to grasp only result D of app D out of result C of app C and result D of app D will be explained. In this case, for the user to grasp result D, result D is necessary to be displayed. Moreover, to display result D, it is necessary to avoid discarding result D. On the other hand, result C is not required to be displayed and result C may be discarded.

Accordingly, the user enters various kinds of information for an item of priority, an item of screen display, and an item of discardability for each of result C and result D by operating the input interface 53. In the item of priority, when the user wishes to be aware of the result, “HIGH” indicating that the user wishes to be aware of the result is set, and when the user does not wish to be aware of the result, “LOW” indicating that the user does not wish to be aware of the result is set. Note that priority may be set for processes instead of results.

Moreover, in the item of screen display, when the user wishes to display the result on a screen, “YES” indicating that the result is to be displayed on a screen is set, and when the user does not wish to display the result on a screen, “NO” indicating that the result is not to be displayed on a screen is set.

Furthermore, in the item of discardability, when the user wishes to discard the result, “YES” indicating that the result is allowed to be discarded is set, and when the user does not wish to discard the result, “NO” indicating that the result is not allowed to be discarded is set.

To explain with specific examples, the user sets “HIGH” indicating that the user wishes to be aware of result D in the item of priority for result D, and sets “YES” indicating that result D is to be displayed on a screen in the item of screen display for result D, and sets “NO” indicating that result D is not allowed to be discarded in the item of discardability for result D by operating the input interface 153. Specifically, the setting function 155a sets “HIGH” indicating that the user wishes to be aware of result D in the item of priority for result D, sets “YES” indicating that result D is to be displayed on a screen in the item of screen display for result D, and sets “NO” indicating that result D is not allowed to be discarded in the item of discardability for result D based on the operation of the user accepted through the input interface.

Moreover, the user sets “LOW” indicating that the user does not wish to be aware of result C in the item of priority for result C, sets “NO” indicating that result C is not to be displayed on a screen in the item of screen display for result C, and sets “YES” indicating that result C is allowed to be discarded in the item of discardability for result C by operating the input interface 153. Specifically, the setting function 155a sets “LOW” indicating that the user does not wish to be aware of result C in the item of priority for result C, sets “NO” indicating that result C is not to be displayed on a screen in the item of screen display for result C, and sets “YES” indicating that result C is allowed to be discarded in the item of discardability for result C based on the operation of the user accepted through the input interface.

“HIGH” and “LOW” set in the item of priority can be regarded as information indicating whether final execution result is necessary. Therefore, in the definition information 152a, information indicating whether it is necessary is set for a final execution result of respective apps included in the workflow.

After the definition information 152a is thus changed, the executing function 155b executes a workflow in accordance with the definition information 152a after a change. For example, in the definition information 152a after a change, “LOW” is set in the item of priority for result C, “NO” is set for the item of screen display for result C, and “YES” is set for the item of discardability for result C. From these settings, it is understood that it is not necessary to execute processes of app C to completion to generate result C.

Moreover, for example, in the definition information 152a after a change, “HIGH” is set in the item of priority for result D, “YES” is set in the item of screen display for result D, and “NO” is set in the item of discardability for result D. From these settings, it is understood that it is necessary to execute process of app D to completion to generate result D.

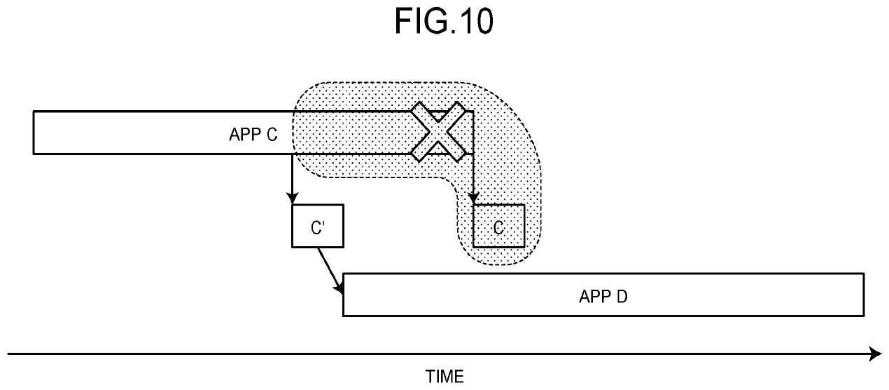

Therefore, as illustrated in FIG. 10, the executing function 155b executes app C until intermediate data C′ of app C to be used in execution of app D is generated, and terminates execution of app C as soon as intermediate data C′ is generated. The executing function 155b inputs intermediate data C′ output from app C to app D, and executes app D using intermediate data C′. Subsequently, result D is output from app D. The display control function 155c displays result D on at least one of the display 154 and the terminal 130. FIG. 10 is a diagram for explaining an example of processing performed by the executing function 155b according to the second embodiment.

That is, when the executing function 155b inputs intermediate data generated by one app C out of multiple apps to another app D, and executes the other app D using the intermediate data, if information indicating that final execution result of one app C is not necessary and information indicating that final execution result of the other app D is necessary are set in the definition information 152a, the executing function 155b executes one app C until one app C generates the intermediate data, terminates execution of app C at the point when the intermediate data is generated, inputs the generated intermediate data to the other app D, and executes the other app D using the input intermediate data.

Moreover, the executing function 155b starts execution of the other app D as soon as execution of one app C is terminated.

As indicated by a range surrounded by a broken line frame in FIG. 10, the executing function 155b does not continue execution of app C unnecessarily after result C is output to app D, and terminates execution of app C. Thus, for example, a processing load on the processing circuitry 155 implemented by a processor or the like is reduced. Moreover, in the storage circuit 152 implemented by a memory or the like, unnecessary data is not stored and, therefore, a memory capacity of the storage circuit 152 does not need to be overused. Therefore, according to the medical data-processing system 100 and the medical data-processing apparatus 150 according to the second embodiment, wasteful consumption of resources of the medical data-processing apparatus 150 can be suppressed.

Furthermore, the executing function 155b starts execution of app D at the point when execution of app C is ended in the middle without waiting completion of execution of app C. Therefore, according to the medical data-processing system 100 and the medical data-processing apparatus 150 according to the second embodiment, over all execution time of a workflow can be reduced.

From the above, according to the medical data-processing system 100 and the medical data-processing apparatus 150 according to the second embodiment, a workflow can be executed appropriately.

Specific examples of app C and app D will be explained. For example, app C is an app that generates a perfusion image by using multiple pieces of chronological three-dimensional CT image data as DICOM data. Moreover, app D is an app that performs predetermined analysis (for example, analysis relating to cerebral infarction) for occlusion of LVO of a subject using result C′. Although app C generates a perfusion image as execution result C, it outputs three-dimensional CT image data with the highest concentration of contrast agent administered to a subject from among multiple pieces of three-dimensional CT image data to app D, as execution result C′ to be output to app D. App D then performs the predetermined analysis of LVO using the three-dimensional CT image data with the highest concentration of contrast agent output from app C, and outputs an analysis result as result D.

When an event of termination of execution of the workflow is detected, the display control function 155c displays result C′ (intermediate data of app C) that is input data input to app D on at least one of the display 154 and the terminal 130.

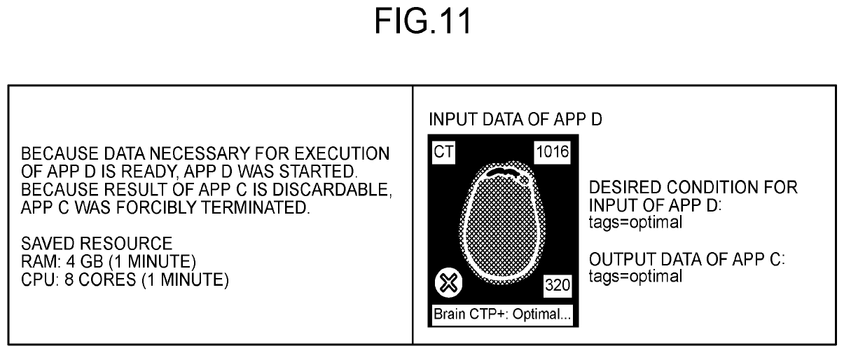

FIG. 11 is a diagram illustrating an example of input data input to app D that is displayed on at least one of the display 154 and the terminal 130 in the second embodiment. As illustrated in FIG. 11, the display control function 155c displays three-dimensional CT image data with the highest concentration of contrast agent administered to a subject as input data input to app D on at least one of the display 154 and the terminal 130. Therefore, the user can easily grasp data input to app D intuitively. Moreover, the user can easily confirm whether intended data is input to app D.

Moreover, by displaying input data input to app D, the user can easily confirm whether proper processing has been performed. This contributes to supporting users in discovering a more efficient workflow.

Furthermore, as illustrated in FIG. 11, the display control function 155c displays an attribute of input data to be used in app D and an attribute of input data that has actually been output from app C and input to app D in a comparative manner on at least one of the display 154 and the terminal 130. In the example in FIG. 11, the display control function 155c displays “optimal” as the attribute of input data to be used in app D. For example, tags are added to a medical image, and for the tags, various kinds of attributes are registered. Furthermore, the display control function 155c displays “optimal” as the attribute of input data that is actually output from app C and input to app D. As described, the display control function 155c displays an attribute of input data to be used in one app (app D) and an attribute of data of another app (app C) on at least one of the display 154 and the terminal 130. By checking the two attributes, the user can easily check whether intended data is input to app D. In the example illustrated in FIG. 11, because the attributes of the two match, the user can confirm that intended data is input to app D easily.

When the attributes of two do not match, the user can determine that unintended data is input to app D easily. In this case, the display control function 155c may display an acceptance screen to accept an instruction from the user to change the workflow or to perform manual re-execution of process in which an error has occurred on at least one of the display 154 and the terminal 130. The medical data-processing apparatus 150 may perform processing in accordance with the instruction accepted from the user.

Moreover, as illustrated in FIG. 11, the display control function 155c displays a message explaining by what cause the execution of app D started and by what cause the execution of app C terminated on at least one of the display 154 and the terminal 130. Specifically, the display control function 155c displays a message “Because data necessary for execution of app D is ready, app D was started. Because result of app C is discardable, app C was forcibly terminated” on at least one of the display 154 and the terminal 130. Thus, the user can easily understand by what cause the execution of app D was started, and by what cause the execution of app C was terminated.

Furthermore, as illustrated in FIG. 11, the display control function 155c displays “Saved Resources: RAM: 4 GB (1 minute), CPU: 8 Cores (1 minute) indicating how much resources have been saved on at least one of the display 154 and the terminal 130. The executing function 155b estimates how much resources have been saved from a past usage amount of resources in past. The display control function 155c then displays the estimated usage amount of resources. That is, the display control function 155c displays an amount of saved resources in the case in which one app C is executed until intermediate data is generated compared to the case in which one app C is executed until a final execution result is generated on at least one of the display 154 and the terminal 130. Thus, the user can easily grasp how much resources have been saved.

Moreover, in the present embodiment, the display control function 155c displays result D that the user wishes to be aware of on at least one of the display 154 and the terminal 130. On the other hand, the display control function 155c does not display result C that the user does not wish to be aware of. Accordingly, by configuring not to display result C that the user does not wish to be aware of, it is possible for the user to comprehend a result efficiently.

As above, the medical data-processing system 100 and the medical data-processing apparatus 150 according to the second embodiment have been explained. According to the medical data-processing system 100 and the medical data-processing apparatus 150 according to the second embodiment, as described above, a workflow can be executed appropriately.

### Third Embodiment

The medical data-processing apparatus 150 may perform processing to change execution order of multiple apps according to an execution state of a workflow. Therefore, such an embodiment will be explained as a third embodiment. In explanation of the third embodiment, a point different from the embodiments described above and the modifications described above will be mainly explained, and explanation of configurations similar to those of the embodiment described above and the modifications described above may be omitted.

In the third embodiment, the executing function 155b executes a workflow including process E, process F, and a summary report. The summary report is an app to summarized result E of process E and result F of process F, and to display summarized content on at least one of the display 154 and the terminal 130. Therefore, the summary report is not an app for a user, such as a physician, to write findings. Prior to such execution of the workflow by the executing function, definition relating to execution of the workflow indicated in the definition information 152a is changed by the user.

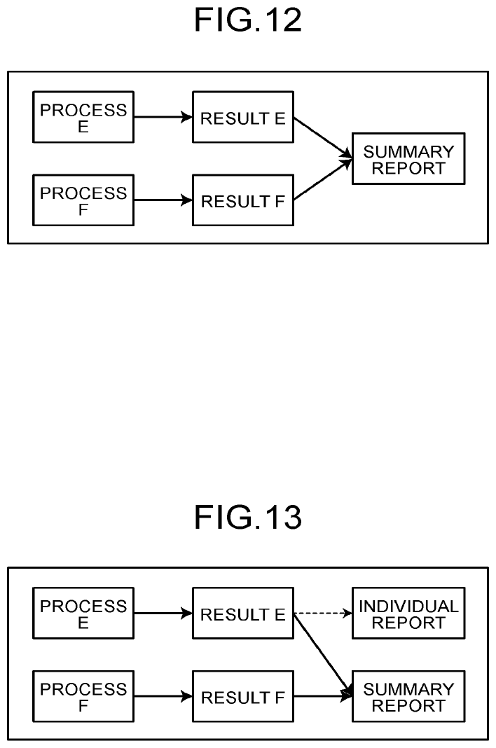

FIG. 12 is a diagram illustrating an example of the definition information 152a before a change according to the third embodiment. As illustrated in FIG. 12, in the definition information 152a before a change, it is defined that process E is executed, and result E of process E is input to the summary report. Moreover, in the definition information 152a before a change, it is defined that process F is executed, and result F of process F is input to the summary report.

In the following, a case in which result E is a result of non-contrast imaging and result F is a result of contrast imaging will be explained. In this case, the summary report displays a report in which results of non-contrast imaging and results of contrast imaging are summarized. Generally, in summary reports, a result of non-contrast imaging is input first, and a result of contrast imaging is input later. When there is a significant time gap between the input of the result of non-contrast imaging and the input of the result of contrast imaging, there is a request of the user to review an individual report in which only the result of non-contrast imaging is described first, and then to review the report in which the result of non-contrast imaging and the result of contrast imaging are collected.

Therefore, to meet the request described above, the user changes the definition information 152a before a change in FIG. 12. FIG. 13 is a diagram illustrating an example of the definition information 152a after a change according to the third embodiment.

As illustrated in FIG. 13, the user sets an arrow toward an individual report from result A such that result A is input to the individual report when result B is not output within a predetermined time (for example, three minutes) since result A is output from process A by operating the input interface 153. The individual report is an app to display an input result converting into a predetermined report format on at least one of the display 154 and the terminal 130. That is, the individual report displays an input result as an individual report. Specifically, the setting function 155a sets an arrow toward the individual report from result A such that result A is input to the individual report when result B is not output from process B within a predetermined time since result A is output from process A based on an operation of a user accepted through the input interface 153. Thus, in the definition information 152a after a change, it is defined that result A is input to the individual report when result B is not output from process B within the predetermined time since result A is output from process A. More specifically, in the definition information 152a after a change, an event in which result B is not output from process B within a predetermined time since result A is output from process A, and input of result A into the individual report are registered in an associated manner.

Moreover, the user adds a definition that result B is input to the summary report when result B is output from process B after the predetermined time passes since result A is output from process A, and result A is also input to the summary report, to the definition information 152a by operating the input interface 153. Specifically, the setting function 155a adds the definition that result B is input to the summary report when result B is output from process B after the predetermined time passes since result A is output from process A, and result A is also input to the summary report, to the definition information 152a based on the operation of the user accepted through the input interface 153. More specifically, in the definition information 152a, an event in which result B is output from process B after the predetermined time passes since result A is output from process A, and input of result A and result B to the summary report are registered in an associated manner.

Furthermore, the user adds a definition that result A and result B are input to the summary report when result B is output from process B within the predetermined time since result A is output from process A, to the definition information 152a by operating the input interface 153. Specifically, the setting function 155a adds the definition that result A and result B are input to the summary report when result B is output from process B within the predetermined time since result A is output from process A, to the definition information 152a based on the operation of the user accepted through the input interface 153. More specifically, in the definition information 152a, an event in which result B is output from process B within the predetermine time since result A is output from process A, and input of result A and result B to the summary report are registered in an associated manner.

After the definition information 152a is thus changed, the executing function 155b executes the workflow in accordance with the definition information 152a after the change. For example, when the event in which result B is not output from process B within the predetermined time since result A is output from process A is detected, the executing function 155b performs processing associated with this event. Specifically, the executing function 155b inputs result A to the individual report. Thus, the user can review an individual report in which only the result from non-contrast imaging is described first.

Moreover, when the event in which result B is output from process B after the predetermined time passes since result A is output from process A is detected, the executing function 155b performs processing associated with this event. Specifically, the executing function 155b inputs result A and result B to the summary report. Thus, the user can review a report in which the result from non-contrast imaging and the result from contrast imaging are collected thereafter.

Furthermore, when the event in which result B is output from process B within the predetermined time since result A is output from process A is detected, the executing function 155b performs processing associated with this event. Specifically, the executing function 155b inputs result A and result B to the summary report. Thus, the user can review a report in which the result from non-contrast imaging and the result from contrast imaging are collected at once.

As above, the medical data-processing system 100 and the medical data-processing apparatus 150 according to the third embodiment have been explained. As described above, the executing function 155b performs processing of changing execution order of multiple apps according to an execution state of a workflow. Therefore, according to the medical data-processing system 100 and the medical data-processing apparatus 150 according to the third embodiment, a workflow can be executed appropriately.

The executing function 155b may perform processing of changing execution order of multiple processes included in a workflow according to an execution state of the workflow by a method similar to the processing of changing execution order of multiple apps according to an execution state of the workflow.

### Fourth Embodiment

In the first embodiment and the like described above, the case in which the medical data-processing apparatus 150 continues to execute a process or an app even when an error occurs has been explained. However, when an error occurs, the medical data-processing apparatus 150 may confirm with a user whether to continue execution of a process or an app. Therefore, such an embodiment will be explained as a fourth embodiment. In explanation of the fourth embodiment, a point different from the embodiments described above and the modifications described above will be mainly explained, and explanation of configurations similar to those of the embodiments described above and the modifications described above may be omitted.

In the fourth embodiment, when an error occurs, the display control function 155c displays an acceptance screen to accept an instruction indicating whether to continue to execute a process or an app from a user on at least one of the display 154 and the terminal 130. On the acceptance screen, a cause of the error is shown. The display control function 155c may transmit the acceptance screen to a terminal used by the user by email, to display the acceptance screen on the terminal. Moreover, the display control function 155c may execute an application to display the acceptance screen, to thereby display the acceptance screen.

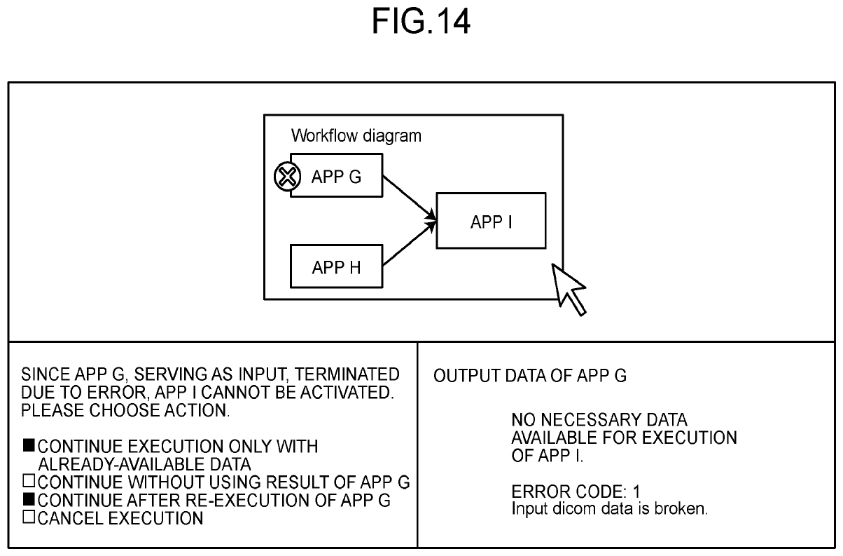

FIG. 14 is a diagram illustrating an example of the acceptance screen according to the fourth embodiment. When an event of occurrence of an error is detected, the display control function 155c displays the acceptance screen illustrated in FIG. 14 on at least one of the display 154 and the terminal 130. The example in FIG. 14 illustrates that an error has occurred in app G out of app G, app H, and app I included in a workflow. In this workflow, app G is executed and an result of app G is input to app I, app H is executed and a result of app H is input to app I, and app I is executed using the result of app G and the result of app H, to output a result of app I.

For example, app G, app H, and app I as above are, an app that generates a DWI using MR data, an app that generates a PWI using MR data, and an app that performs mismatch analysis to acquire a mismatch between the DWI and the PWI using the DWI and PWI generated by these two apps.

On the acceptance screen illustrated in FIG. 14, it is indicated that output data (data input to app I and used in app I) that is output from app G is broken, as a cause of the error.

Moreover, on the acceptance screen illustrated in FIG. 14, out of four instruction candidates indicating, whether to continue app executing only with already-available data, whether to continue without using a result of app G, whether to continue after re-execution of app G, and whether to cancel execution of app I, two instruction candidates narrowed down according to the cause of the error are displayed as selectable by the user.

For example, the display control function 155c narrows down to two instruction candidates (to continue without using a result of app G, to cancel execution) having relatively high possibilities of success from among the four instruction candidates according to the cause of an error, and displays the narrowed-down two candidate instructions as selectable by the user. As described, from multiple instruction candidates, the display control function 155c narrows down to executable instruction candidates according to the cause of an error. In the example in FIG. 4, app I is capable, in addition to the functions described above, of generating and outputting a result of app I using only a result of app H, without using a result of app G. Therefore, the display control function 155c displays “CONTINUE WITHOUT USING RESULT OF APP G” as selectable by the user, as an instruction candidate. As described, when an error occurs during execution of a workflow, the display control function 155c accepts an instruction whether to execute subsequent processes. The display control function 155c is one example of an accepting unit. A process based on an accepted instruction may automatically be executed by the executing function 155b as a default for future occurrence of the event.

The executing function 155b controls execution of the workflow based on an instruction selected by the user. For example, when an instruction selected by the user is “CONTINUE WITHOUT USING RESULT OF APP G”, the executing function 155b executes app I using only a result of app H without using a result of app G, and outputs a result of app I. Furthermore, when an instruction selected by the user is “CANCEL EXECUTION”, the executing function 155b terminates processes of the workflow without executing subsequent processes. As described, the executing function 155b controls execution of the workflow. The executing function 155b that controls execution of a workflow as described is one example of a control unit.

As above, the medical data-processing system 100 and the medical data-processing apparatus 150 according to the fourth embodiment has been explained. As described above, the executing function 155b controls execution of a workflow based on an instruction selected by the user. Therefore, according to the medical data-processing system 100 and the medical data-processing apparatus 150 according to the fourth embodiment, a workflow can be executed appropriately to meet a request of a user.

A term “processor” used in the explanation of the embodiments described above signifies a circuit, such as a central processing unit (CPU), a graphical processing unit (GPU), an application specific integrated circuit (ASIC), a programmable logic device (for example, simple programmable logic device (SPLD), complex programmable logic device (CPLD)), and a field programmable gate array (FPGA). Instead of storing a program in the storage circuit 152, it may be configured to directly install a program in a circuit of the processor. In this case, the processor reads and executes the program installed in the circuit, to implement the function. The respective processors of the respective embodiments are not limited to be configured as a single circuit for each processor, but may be configured by combining multiple independent circuits as one processor, to implement its function.

The program executed by the processor is installed in a read only memory (ROM), a storage circuit, or the like in advance to be provided. This program may be provided being stored in a non-transitory computer-readable storage medium that can be read by a computer, such as a compact disk (CD)-ROM, a flexible disk (FD), a compact disk recordable (CD-R), a digital versatile disk (DVD), in a file of a format that can be installed in or executed by these apparatuses. Moreover, this program may be stored in a computer connected to a network, such as the Internet, and may be provided or distributed by being downloaded through the network. For example, this program is constituted of modules including the respective processing functions described above. As actual hardware, a CPU reads and executes the program from the storage medium, such as a ROM, and the respective modules are loaded on a main storage device, and are generated on the main storage device.

According to at least one of the embodiments explained above, a workflow can be executed appropriately.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical data-processing apparatus comprising:
a memory that stores definition information in which input data to be input and output data to be output are set for each execution state of a workflow, for each of a plurality of analysis applications included in the workflow, or for each of a plurality of processes executed in each of the analysis applications; and
processing circuitry that executes the workflow, and performs at least either one of processing of switching a source of either one of input data to be input to one analysis application out of the analysis applications, and input data to be input to one process out of the processes, and processing of changing either one of execution order of the analysis applications and execution order of the processes, according to an execution state of the workflow based on the definition information.

2. The medical data-processing apparatus according to claim 1, wherein
the processing circuitry switches the source of the input data to be input to the one analysis application, and inputs intermediate data of another analysis application to the one analysis application as the input data.

3. The medical data-processing apparatus according to claim 1, wherein
the processing circuitry switches the source of the input data to be input to the one process, and inputs, to the one process, intermediate data of another analysis application that is a different application from an analysis application that executes the one process, as the input data.

4. The medical data-processing apparatus according to claim 1, wherein
the processing circuitry switches the source of the input data to be input to the one process, and inputs, to the one process, data of another process that is a different process from the one process as the input data.

5. The medical data-processing apparatus according to claim 1, wherein
the processing circuitry further sets the input data and the output data depending on an execution state of the workflow, to the definition information.

6. The medical data-processing apparatus according to claim 1, wherein
the processing circuitry further performs displaying the workflow after a change obtained after the source is switched on a display.

7. The medical data-processing apparatus according to claim 1, wherein
the processing circuitry further performs displaying either one of the input data that is input to the one analysis application, and the input data input to the one process, on a display after the source is switched.

8. The medical data-processing apparatus according to claim 4, wherein
the processing circuitry further displays an attribute of input data used for the one process, and an attribute of data of the other process on a display.

9. The medical data-processing apparatus according to claim 1, wherein
the processing circuitry changes the workflow, when an error in execution of the workflow occurs as the execution state of the workflow, by switching the source of the input data to be input to the one analysis application to a source in an event of occurrence of the error, or by switching the source of the input data to be input to the one process to a source in an event of occurrence of the error based on the definition information, and
displays a cause of the change of the workflow, and changes made to the workflow on a display.

10. A medical data-processing apparatus comprising:
a memory that stores definition information in which input data to be input to a process executed in an analysis application included in a workflow is set for each execution state of the workflow; and
processing circuitry that executes the workflow, and performs processing of switching input data to be input to the process according to an execution state of the workflow based on the definition information.

11. The medical data-processing apparatus according to claim 10, wherein
in the definition information, first input data is set as input data to be input to the process when the execution state of the workflow is a first execution state, and second input data replaceable with the first input data is set as input data to be input to the process when the execution state of the workflow is a second execution state, and
the processing circuitry executes the process by inputting the first input data to the process when the execution state of the workflow is the first execution state, and executes the process by inputting the second input data to the process when the execution state of the workflow is the second execution state.

12. The medical data-processing apparatus according to claim 10, wherein
in the definition information, a plurality of pieces of the input data are set for each execution state of the workflow for the process, and priorities corresponding to each of the pieces of the input data are further set, and
the processing circuitry switches input data to be input to the process from among the pieces of the input data according to the priority.

13. A medical data-processing apparatus comprising:
a memory that stores definition information in which information indicating whether a final execution result is necessary for each of a plurality of analysis applications included in a workflow is set; and
processing circuitry that executes the workflow based on the definition information, wherein
when the processing circuitry inputs intermediate data generated by one analysis application out of the analysis applications to another analysis application, and executes the other analysis application using the intermediate data, if information indicating that a final execution result of the one analysis application is not necessary and information indicating that a final execution result of the other analysis application is necessary are set in the definition information, the processing circuitry executes the one analysis application until the intermediate data is generated by the one analysis application, and terminates the execution of the one analysis application at a point of time when the intermediate data is generated, inputs the generated intermediate data to the other analysis application, and executes the other analysis application using the input intermediate application.

14. The medical data-processing apparatus according to claim 13, wherein
the processing circuitry starts execution of the other analysis application at a point of time when execution of the one analysis application is terminated.

15. The medical data-processing apparatus according to claim 13, wherein
the processing circuitry further performs displaying a saved amount of resource when the one analysis application is executed until the intermediate data is generated compared to a case in which the one analysis application is executed until a final execution result is generated, on a display.

16. A medical data-processing apparatus comprising:
processing circuitry that executes a workflow including a plurality of analysis applications, that accepts an instruction whether to execute a subsequent process when an error occurs during execution of the workflow, and that controls execution of the subsequent process based on the accepted instruction.

17. A system comprising:
a memory that stores definition information in which input data to be input and output data to be output are set for each execution state of a workflow, for each of a plurality of analysis applications included in the workflow, or for each of a plurality of processes executed in each of the analysis applications; and
processing circuitry that executes the workflow, and performs at least either one of processing of switching a source of either one of input data to be input to one analysis application out of the analysis applications, and input data to be input to one process out of the processes, and processing of changing either one of execution order of the analysis applications and execution order of the processes, according to an execution state of the workflow based on the definition information.

18. A system comprising:
a memory that stores definition information in which input data to be input to a process executed in an analysis application included in a workflow is set for each execution state of the workflow; and
processing circuitry that executes the workflow, and performs processing of switching input data to be input to the process according to an execution state of the workflow based on the definition information.

19. A system comprising:
a memory that stores definition information in which information indicating whether a final execution result is necessary for each of a plurality of analysis applications included in a workflow is set; and
processing circuitry that executes the workflow based on the definition information, wherein
when the processing circuitry inputs intermediate data generated by one analysis application out of the analysis applications to another analysis application, and executes the other analysis application using the intermediate data, if information indicating that a final execution result of the one analysis application is not necessary and information indicating that a final execution result of the other analysis application is necessary are set in the definition information, the processing circuitry executes the one analysis application until the intermediate data is generated by the one analysis application, and terminates the execution of the one analysis application at a point of time when the intermediate data is generated, inputs the generated intermediate data to the other analysis application, and executes the other analysis application using the input intermediate application.

20. A system comprising:
processing circuitry that executes a workflow including a plurality of analysis applications, that accepts an instruction whether to execute a subsequent process when an error occurs during execution of the workflow, and that controls execution of the subsequent process based on the accepted instruction.

21. A method comprising:
executing a workflow based on definition information in which input data to be input and output data to be output are set for each execution state of the workflow, for each of a plurality of analysis applications included in the workflow, or for each of a plurality of processes executed in each of the analysis applications; and
performing processing of switching a source of either one of input data to be input to one analysis application out of the analysis applications, and input data to be input to one process out of the processes, and processing of changing either one of execution order of the analysis applications and execution order of the processes, according to an execution state of the workflow.

22. A method comprising:
executing a workflow based on definition information in which input data to be input to a process executed in an analysis application included in the workflow is set for each execution state of the workflow; and
performing processing of switching input data to be input to the process according to an execution state of the workflow.

23. A method comprising:
executing a workflow based on definition information in which information indicating whether a final execution result is necessary for each of a plurality of analysis applications included in the workflow is set; and
when intermediate data generated by one analysis application out of the analysis applications is input to another analysis application, and the other analysis application is executed using the intermediate data, if information indicating that a final execution result of the one analysis application is not necessary and information indicating that a final execution result of the other analysis application is necessary are set in the definition information, executing the one analysis application until the intermediate data is generated by the one analysis application, terminating the execution of the one analysis application at a point of time when the intermediate data is generated, inputting the generated intermediate data to the other analysis application, and executing the other analysis application using the input intermediate application.

24. A method comprising:
executing a workflow including a plurality of analysis applications;
accepting an instruction whether to execute a subsequent process when an error occurs during execution of the workflow; and
controlling execution of the subsequent process based on the accepted instruction.
